(19) United States
(12) Patent Application Publication
(10) Pub. No.: US 2025/0302571 A1
(43) Pub. Date: Oct. 2, 2025

(54) Releasable Closure For a Sharps Bin

(71) Applicant: INPRESS PRECISION LTD, Littleham, Sussex (GB)

(72) Inventors: Henry Powell, Littleham (GB); William Powell, Littleham (GB); Tony BEESON, Littleham (GB); Justas ZELVYS, Littleham (GB); John MCINERNEY, Littleham (GB); Kieron KING, Littleham (GB); Ben HUNTER, LittlehamG (GB)

(21) Appl. No.: 19/118,124

(22) PCT Filed: Aug. 11, 2023
(86) PCT No.: PCT/GB2023/052134

§ 371 Date: Apr. 3, 2025

(30) Foreign Application Priority Data
Aug. 18, 2022 (GB) 2212051.3

(51) Int. Cl. A61B 50/36; A61B 50/00; B67B 7/14

(52) U.S. Cl. CPC A61B 50/362; B67B 7/14; A61B 2050/0051; A61B 2050/0084

(57) ABSTRACT

A system that allows for the re-use of a sharps locked bin. The system comprises a bin for medical waste comprising a main body with a peripheral flange and a lid with an outer rim. When the bin is locked, a gap is located between an internal rim of the flange of the main body and the outer rim of the lid. The system also comprises bin opening apparatus comprising a base jig and a pillar drill which includes a plate orientated generally horizontally in use and pronged released members extending downwardly from the plate.

### FIELD OF THE INVENTION

The present invention relates to a releasable closure. More especially, the invention relates to a closure for a sharps bin used to capture, for example, medical waste. The invention also relates to a method of releasing the locked closure.

### BACKGROUND

The present invention is an improvement to a closure for a sharps bin described in GB2369109B, a patent acquired by the applicant.

Traditionally, once closures for sharps bins, such as the one described in the patent, are locked it is not possible to unlock the closure or remove the lid in order to prevent access to the contents. The sharps bin is transported to a medical incinerator where the entire bin and its contents are incinerated. Sharp bins have therefore been designed specifically for single use.

Whilst the sharps bin of the patent has proven to be commercially successful, recent changes in attitudes caused by worldwide environmental issues have led to a need for the bin of this type to be reusable in order to reduce the environmental impact on the planet.

Furthermore, sharps bins that are transported to medical incinerators are rarely full, often they are only half full. The transport costs for the medical waste placed in the bins can be greatly decreased if only the contents of the bins, and not the bins themselves, are required to be transported to the medical waste disposal facility.

The present inventions seeks to provide an improved sharps bin with a lid and closure that is lockable during use, but which can released from the main body of the bin using a specific method and closure release tool to allow the main body to be re-used.

The present invention seeks to provide a system for providing a reusable sharps bin to the medical industry with means to open the bin in a secure environment and to treat and provide at least part of the bin back to the industry for re-use.

The present invention also provides apparatus and a method for opening the bin in the secure environment to allow for its re-use.

### BRIEF DESCRIPTION OF FIGURES

A preferred embodiment of the invention will now be described by way of example only and with reference to the Figures in which:

FIG. 1 is a sharps container that forms part of the state of the art;

FIG. 2 is a closeup view of the connection between the lid and the main body;

FIG. 3 illustrates a release device used to separate the lid of the container from the main body;

FIGS. 4 and 5 illustrate the device acting to release the lid from the main body;

FIG. 6 illustrates the release members of the device; and

FIG. 7 further illustrates the release member acting to release the lid from the main body.

### DETAILED DESCRIPTION OF ONE EMBODIMENT OF THE INVENTION

The present invention relates primarily to a method of releasing a locked lid and closure from a sharps bin.

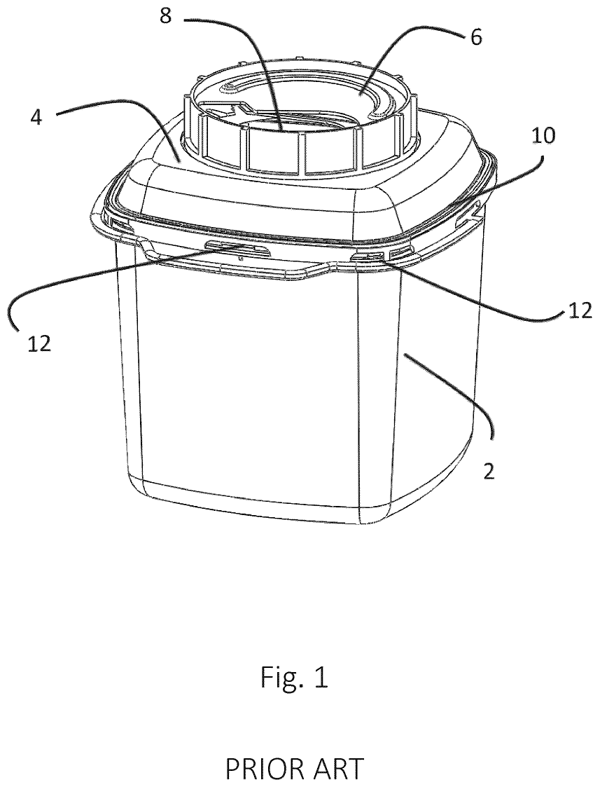

FIG. 1 shows a prior art sharps bin of the type described in GB2369109B. It generally comprises a main body 2 with a lid 4 adapted to be removably connected to the main body 2 during use, and which is locked in place once the main body 2 is filled.

The main body 2 has a circular upstanding central region (not shown) in which there is a large access aperture (also not shown) having formations for use in detaching needles from hypodermic syringes. A closure member 6 has the form of an inverted cup with an outer radial flange around its mouth. This fits rotatably over the upstanding region, and the mouth engages in an annular channel in the lid 4, around the base of the upstanding region.

In the base of the closure member 6, there is an aperture 8 which is shaped and dimensioned so that it can be placed in register with the access aperture to allow full access to and through that aperture.

The closure member 6 can be rotated to close the aperture and, once the main body 2 is full or its use is otherwise no longer required, the closure member 6 is irreversibly locked to prevent access to the contents of the bin. Access to the contents of the bin is also prevented by the locking connection between the main body 2 and the lid 4.

Ordinarily the entire sharps bin would then be taken to a medical incinerator where the bin and its contents are incinerated.

In order to ensure that the contents of the sharps bin are not accessible, the main body 2 and lid 4 are locked together and, ordinarily, cannot be removed from one another.

The locking mechanism is provided by the interlocking features formed integral to the lid and bin edges. The top edge of the bin 2 is formed with a peripheral flange 10 having a depth to accommodate elongated slots or apertures 12 formed at each corner and generally centrally along each side.

The bottom edge of the lid 4 has corresponding wedge-shaped locking members 14 (seen in FIG. 7) designed to protrude outwardly through the apertures 12 when the lid 4 is locked in place.

The bin 2 is made from plastics material providing some inherent elasticity to the bin structure thereby to allow the main body 2 to flex as the lid 4 is placed onto the main body 2 and the locking members 14 move downwardly against the inside of the flange 10 to be received into and through the apertures 12.

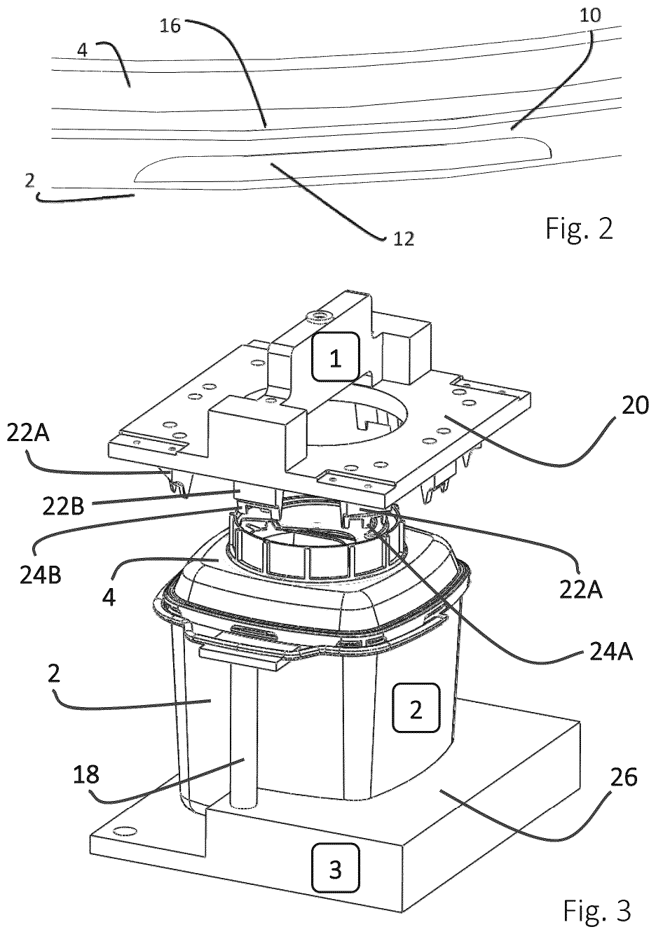

As can be seen in FIG. 2, a gap 16 is formed between the internal rim of flange of the main body 2 and the outer rim of the lid 2, above each aperture 12.

In order to meet the requirements for the bin to be recyclable and reusable, the applicant has developed a method of releasing the lid 4 from the main body 2 using the release tool.

The tool is designed for industrial use and neither the tool nor the method are appropriate for use by untrained personnel, to ensure that the bin contents remain inaccessible during the environment of their use, for example in a hospital and whilst the bin is being transported to the release premises.

The lid 4 can only be released from the main body 2 by authorised trained personnel and at authorised premises where the conditions meet the required strict standards for medical waste disposal.

One released, the contents can be removed for incineration and the main body 2 can be medically cleaned for re-use with another lid 4 and closure 6.

After the release process, the lid 4 and closure 6 is re-ground to provide re-cycled material for the production of new lids 4 and closures 6.

FIGS. 3 to 5 illustrate the apparatus and method for releasing the lid 4 from the main body 2.

The release device is shown in FIG. 3. Only the key features of the device needed for the purpose of understanding the invention, will now be described.

The removal device is attached a pillar drill 18 and includes a horizontally orientated plate 20 from which a plurality of multiple pronged release members 22 with the prongs 24 orientated downwardly from the plate 20 in use.

The release members 22 are located in shape and formation that correspondences to the shape of the bin 2 such that, when the bin 2 is placed on a base jig 26 directly underneath the plate 20, and the plate 20 is lowered, the release members 22 engage with the main body 2 and lid 4 and act to push the prongs 24 downwardly into and through the gap 16 between the internal rim of the flange 10 of the main body 2 and the outer rim of the lid 4.

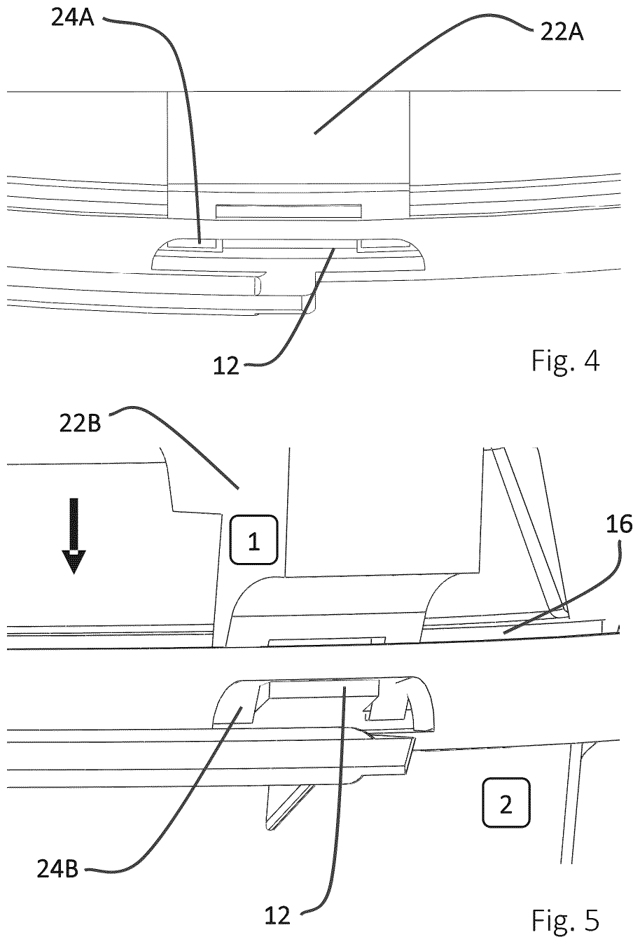

FIGS. 4 and 5 show the prongs 24 of each release member 22 extending into the gap 16.

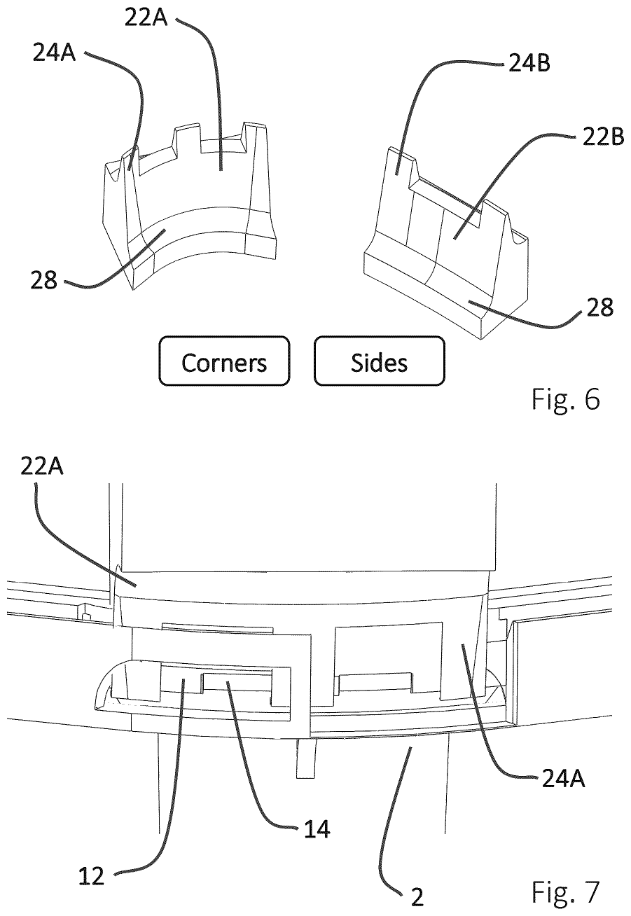

FIG. 6 shows a corner release member 22A and a side release member 22B. The release members 22 are formed from a strong robust metal. The corner release member 22A has a generally curved construction whereas the side release member 22B has a generally flat form.

The corner release member 22A has three prongs 24A whereas the side release member 22B has only two prongs 24B.

Both members are generally tapered outwardly from the tips of the prongs 24 to the base 28 from which they extend.

The taper provides a gentle increase in the flexing of the flange 10 as the prongs 24 are pushed down into the gap 16 until such point where the gap 16 is sufficiently wide to release the locking members 14 from their respective apertures 12 to allow the lid 4 to be released by lifting it from the main body 2.

In the embodiment described there are eight release members 22 to be received in eight apertures 12 in the flange associated with the wedge-shaped members of the lid 4. The number, shape and/or position of the release members 22 may vary depending on the number, shape and/or position of the wedges 14 to be released.

The wedged locking member 14 can be seen in FIG. 7. It is appreciated that the flange 10 has been cut away in this figure only for the purposes of showing the prongs 24A of the release member 22A.

In order to release the lid 4 from the main body 2, all the release members 22 must act simultaneously to release each wedge 14 from its respective aperture 12.

Once released, the contents of the bin 2 are placed or transported to medical waste disposal facility and the main body 2 is medically cleaned for re-use.

The lid 4 is ground down to provide recycled plastic material for further lids and closures. To this end, the lid and closure mechanism is formed solely of recycled plastic material. The closure does not have any springs, for example, as those described in GB2369109B. An alternative closure locking mechanism may be used which does not utilise any components of an alternative material.

The main body 2 may include RFID technology and/or a surface-printed bar code that can be scanned at the release facility so that the number of uses of the bin can be tracked and monitored to avoid any issues with degradation over time and/or the number of times a particular bin has been cleaned and recycled.

Main bodies 2 can be stacked for storage and assembled with a lid on site when ready.

It is anticipated that recycling of sharps bins in form of the method and system described would reduce the provide a virtual loop of plastic recycling and reduce the carbon footprint of the sector by as much as 25%.

1. A system that allows for the re-use of a locked bin, the system comprising
a bin for medical waste comprising:
a main body with a peripheral flange; and
a lid with an outer rim;
wherein, when the bin is locked, a gap is located between an internal rim of the flange of the main body and the outer rim of the lid;
bin opening apparatus comprising:
a base jig for receiving a bin in use;
a plate orientated generally horizontally in use; the plate having two or more pronged released members extending from the plate; and
means to move the plate vertically towards the bin to locate the release members into the gap between the main body and lid of the bin.

2. A system according to claim 1, wherein the plate movement means comprises a pillar drill.

3. A system according to claim 1, wherein the bin has a locking mechanism provided by interlocking members formed integral with edges of the main body and lid respectively.

4. A system according to claim 3, wherein the interlocking feature of the main body comprises elongate slots formed in each corner and along each side of the flange.

5. A system according to claim 4, wherein the interlocking feature of the lid comprises wedge-shaped locking members shaped to be received within respective slots in the flange.

6. A system according to claim 1, wherein the main body and lid are made from plastics material.

7. A system according to claim 1, wherein the pronged release members of the bin opening apparatus comprising at least one corner release member and at least one side release member.

8. A system according to claim 7, wherein the or each corner release member has a generally curved profile.

9. A system according to claim 8, wherein the or each side release member has a generally flat profile.

10. A system according to claim 7, wherein the or each corner release member has three prongs.

11. A system according to any one of claims 7 to 10, wherein the or each side release member has two prongs.

12. A system according to any one of claims 7 to 11, where the release members are tapered outwardly from the tops of the prongs to a base from which the prongs extend.

13. A system according to claim 1, wherein the bin opening apparatus has eight pronged release members to be received within eight apertures in the flange of the main body.

14. A system according to claim 1, wherein the main body includes a scannable bar code to identify the number of occasions the bin has been opened by the bin opening apparatus.

15. A system according to claim 1, wherein the main body includes an RFID chip to monitor the number of occasions the bin has been opened by the bin opening apparatus.

16. A method of opening a locked bin using the system according to claim 1, the method comprising the steps of:
placing the locked bin on the base jig directly underneath the plate;
activating the pillar drill to lower the plate such that the pronged release members extend downwardly into the gaps to thereby separate the lid from the main body.

17. A method according to claim 16, further comprising re-grounding the lid, once separated from the main body, to provide re-cycled material for the production of a new lid.

18. A method according to claim 17, further comprising the step of removed the contents of the bin once the lid and main body have been separated, and medically cleaning the main body for re-use with another lid.
